(19) United States
(12) Patent Application Publication
(10) Pub. No.: US 2004/0215132 A1
(43) Pub. Date: Oct. 28, 2004

(54) Spot coagulating & occluding instrument and method of use

(76) Inventor: InBae Yoon, Ellicott City, MD (US)

Correspondence Address: ROYAL W. CRAIG, A PROFESSIONAL CORPORATION, SUITE 153, 10 NORTH CALVERT STREET, BALTIMORE, MD, 21202, US

(21) Appl. No.: 10/421,383

(22) Filed: Apr. 22, 2003

(51) Int. Cl. A61M 31/00

(52) U.S. Cl. 604/57

(57) ABSTRACT

A spot-occlusion apparatus spot-coagulates or otherwise occludes an anatomical tissue structure which is disposed in a living body and has a plurality of vessels extending therefrom. The occlusion apparatus includes an elongated tubular member and an occluding mechanism. The tubular member extends along a central longitudinal axis to define a lumen and has a distal end positioned interiorly of the living body and a proximal end disposed opposite the distal end and positioned exteriorly of the living body. The occluding mechanism is operative at the distal end of the tubular member and includes a pair of articulating fingers with distal end effectors (such as occluding elements) disposed opposite one another. The pair of end effectors are moveable to and between an opened state and a closed state. In the opened state, the vessels to be occluded are received between the pair of occluding end effectors. In the closed state, the pair of occluding end effectors contact and occlude the vessels, thereby occluding the anatomical tissue structure. The articulating fingers create a working space between themselves for manipulating, coagulating and/or otherwise occluding tissue. Moreover, one or more ports is directed through the lumen into the workspace for admission of a viewing system, hydration device, cauterization device, cryo (cooling) device, suction, or any of a range of ancillary surgical implements all through a single surgical site.

### CROSS-REFERENCE TO RELATED APPLICATIONS

The present application derives priority from U.S. application Ser. No. 09/606,242, filed Jun. 29, 2000.

### BACKGROUND OF THE INVENTION

1. Field of the Invention

This invention relates generally to surgical devices and, more particularly, to an improved surgical apparatus with a pincer ability to reach around healthy tissue to the target area, or with a space-creating ability, as well as a method for using the instrument for spot-coagulation and occlusion of vessels extending from anatomical tissue structures.

2. Description of the Background

A hysterectomy is surgical removal of the uterus resulting in sterility of a female. Typically, hysterectomies may be performed through an abdominal incision, commonly referred to as an abdominal hysterectomy, or through a vaginal incision, commonly referred to as a vaginal hysterectomy. The uterus can be completely removed or partially removed. Also, the uterus can be removed with the fallopian tubes and ovaries. Complications may occur in diagnostic or operative hysterectomy as a result of the surgery, such as uterine perforation, hemorrhage, or delayed complications such as infection or adhesion formation.

To reduce complication there has been a move toward laparoscopic-assisted hysterectomies. This is largely because of the smaller incisions, faster recovery time, and less post-operative pain as compared to a conventional hysterectomy. An endoscope (or other viewing system) is used to allow the surgeon to watch what he or she is doing on video monitors. Most laproscopic hysterectomies require two to three punctures, two for the surgical instrument(s) and one for the endoscope. A separate puncture is required for the viewing system because the surgical site is typically viewed from an angle (the surgical instrument blocks any direct view from the same puncture). Thus, even laproscopic-assisted hysterectomies are somewhat invasive and can result in complications.

Another non-surgical approach to causing sterility of a female entails spot coagulation, which begins with a puncture to the pelvic area for access to the anatomical structure. Given penetration and access to the uterus, the blood vessels of the uterus are spot-coagulated to leave the uterus non-functioning. However, clear access to the blood vessels of the uterus is usually blocked by tissue structures which front the uterus. The close proximity complicates this procedure inasmuch as the tissue structures must be circumnavigated to gain access to the uterine arteries, usually in poor (crowded viewing conditions), and at the risk of inadvertent damage to neighboring tissue structures.

The present invention is an improved surgical apparatus with a pincer ability to reach around healthy tissue to the target area, as well as a method for using the instrument for spot-coagulation and other surgical procedures. The device excels in close-quarter procedures such as occlusion (the alternative surgical procedure to a hysterectomy) because of pincer-like fingers that straddle and reach around healthy tissue. When not reaching around healthy tissue, the articulating fingers create a working space for manipulating, coagulating and occluding targeted tissue. One or more ports pass through the center of the device and enter the working space for admission of a viewing system, hydration device, cauterization device, cryo (cooling) device, or any of a range of ancillary surgical implements all to the single surgical site. The integral viewing system port eliminates the need for a separate puncture for the endoscope. Thus, the apparatus and method reduces surgery time and, because it is less invasive, a patient's hospital stay and recovery period is minimized.

### SUMMARY OF THE INVENTION

Accordingly, it is a principal object of this invention to provide an occlusion apparatus and a method for occluding anatomical tissue structures for use as an alternative surgical procedure to a hysterectomy.

Another object of the invention is to provide an occlusion apparatus and a method for occluding anatomical tissue structures as an alternative surgical procedure for treatment of a variety of organs, such as a uterus, a gall bladder and a prostate.

Another object of the present invention is to provide a combination surgical device with articulating fingers for creating a working space and manipulating, coagulating and occluding tissue, and which additionally includes one or more ports through the same central channel for admission of a viewing system, hydration device, cauterization device, cryo (cooling) device, or any of a range of ancillary surgical implements all through a single surgical site.

Yet another object of the present invention is to provide an occlusion apparatus and a method for occluding anatomical tissue structures that result in reduced hospital stay for the patient.

A still further object of the invention is to provide an occlusion apparatus and a method for occluding anatomical tissue structures that result in reduced recovery time for the patient.

In accordance with the above objects, the present invention is a method for occluding anatomical tissue structures and an occlusion apparatus. The method of the invention necrotizes anatomical tissue structures in a living body. The anatomical tissue structure is connected to a plurality of vessels that extend from the anatomical tissue structure and convey fluids into and out of the anatomical tissue structure. The method of the invention includes locating the anatomical tissue structure to be necrotized and the vessels extending therefrom in the living body. Also, the method of the invention includes occluding the vessels to prevent fluid flow into and out of the anatomical tissue structure to cause ischemic necrosis of the anatomical tissue structure. The occlusion apparatus of the invention spot-coagulates the anatomical tissue structure in the living body. The occlusion apparatus of the invention includes an elongated tubular member and an occluding mechanism. The elongated tubular member extends along a central longitudinal axis to define a lumen. The elongated tubular member also has a distal end positioned interiorly of the living body and a proximal end disposed opposite the distal end and positioned exteriorly of the living body.

The occluding mechanism is operative at the distal end of the tubular member and includes a pair of occluding elements disposed opposite one another. The pair of occluding elements are moveable to and between an opened state and a closed state. In the opened state, the vessels to be occluded are received between the pair of occluding elements. In the closed state, the pair of occluding elements also define a surgical workspace. The tubular member has one or more central ports directed into the workspace (through the same puncture) for admission and viewing by a viewing system, ingress of a hydration device, cauterization device, cryo (cooling) device, or any of a range of ancillary surgical implements all through a single surgical site.

Other objects and advantages of the invention will become apparent from the following description of the embodiments taken in conjunction with the accompanying drawings.

### BRIEF DESCRIPTION OF THE DRAWINGS

Further and more complete objects and advantages of the invention will become readily apparent by reference to the detailed specification and drawings in which:

FIG. 1 is a perspective view of the occlusion apparatus of the invention with facially-opposing occluding end effectors in a spaced-apart opened state.

FIG. 2 is a perspective view of the occlusion apparatus of the invention shown in FIG. 1 with the facially-opposing occluding end effectors in a contacting closed state.

FIG. 3 is an enlarged partial perspective view ofa distal portion of the occlusion apparatus of the invention taken along line 3-3 in FIG. 2.

FIG. 4 is an enlarged partial side view shown partially in cross-section of the occlusion apparatus as shown in FIG. 1.

FIG. 5 is an enlarged partial side view shown partially in cross-section of the occlusion apparatus as shown in FIG. 2.

FIG. 6 is a side view shown partially in cross-section of the occlusion apparatus of the invention shown in FIG. 1.

FIG. 7 is an enlarged partial side view shown partially in cross-section of a proximal end of the occlusion apparatus of the invention.

FIG. 8 is a cross-sectional view of the occlusion apparatus of the invention taken along line 8-8 in FIG. 6.

FIG. 9 is a partial perspective view of an occluding finger having a flattened occluding end effector.

FIG. 10 is a partial perspective view of an alternative occluding finger having a flattened, curved occluding end effector.

FIG. 11 is a partial perspective view of an alternative occluding finger with a tapered section.

FIG. 12 is a side view, partially in cross-section, with the occluding end effectors connected to respective tapered sections and being in the opened state.

FIG. 13 is a side view, partially in cross-section, with the occluding end effectors connected to respective tapered sections and being in the closed state.

FIG. 14 is a diagrammatic view illustrating a pair of occlusion apparatuses of the invention with occluding end effectors occluding vessels of a uterus.

FIG. 15 is an enlarged partial side view of the occluding end effectors occluding a vessel as taken along line 15-15 in FIG. 14.

FIG. 16 is a diagrammatic view of the occlusion apparatus of the invention occluding vessels of a prostate.

FIG. 17 is a diagrammatic view of the occlusion apparatus of the invention occluding a gall bladder.

FIG. 18 is a perspective view of the occluding end effectors used with a representative needle and suture device.

FIG. 19 is a side elevational view of anatomical tissue occluded by tied sutures.

FIG. 20 is an enlarged partial perspective view of The pair of occluding end effectors used and represented as an anvil and stapler device.

FIG. 21 is a side elevational view of anatomical tissue occluded by a staple.

FIG. 22 is an enlarged partial perspective view of the pair of occluding end effectors used represented in combination with a conventional clamp.

FIG. 23 is a side elevational view of the clamp occluding anatomical tissue.

FIG. 24 is an enlarged partial perspective view of the pair of occluding end effectors used with a conventional clip (with side illustration of the clip occluding anatomical tissue).

FIG. 25 is an enlarged partial side view shown partially in cross-section of the occlusion apparatus in an open position substantially as shown in FIG. 1 but without protruberances 30.

FIG. 26 is an enlarged partial side view shown partially in cross-section of the occlusion apparatus as shown in FIG. 25 in closed position.

### DETAILED DESCRIPTION OF THE PREFERRED EMBODIMENTS

An occlusion apparatus of the invention and a method for spot-occlusion of anatomical tissue structures are hereinafter described. The invention is described in the context of spot-occlusion of areas of anatomical tissue structures such as a uterus, a gall bladder and a prostate. However, one of ordinary skill in the art would appreciate that these anatomical tissue structures are used by way of example only, and that other types of anatomical tissue structures, such as a cystic tumor, a kidney, a pancreas and an ovary, can benefit from the invention. Furthermore, one of ordinary skill in the art would appreciate that even portions of a liver or other organs can be spot-coagulated and occluded as described herein.

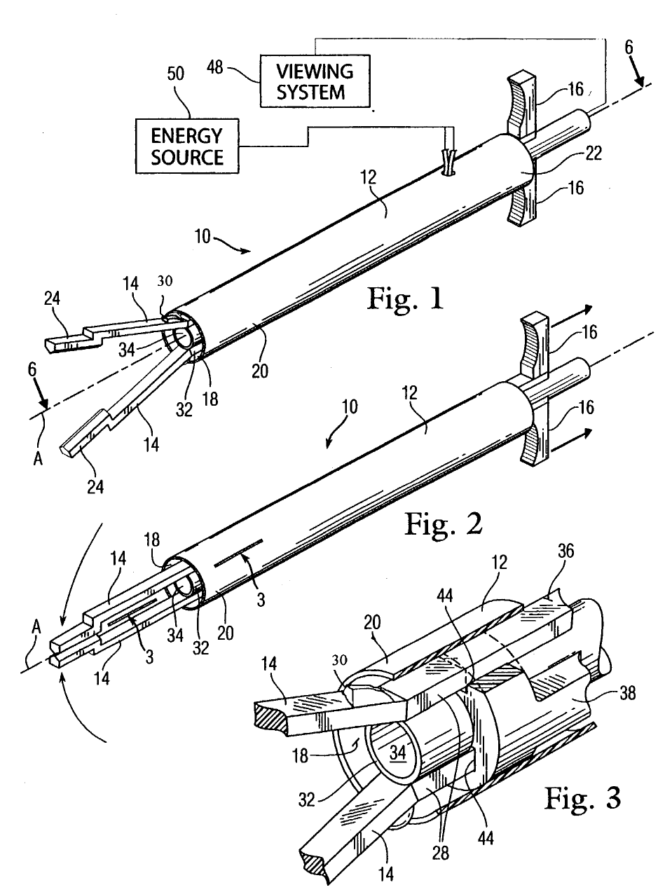

Referring now to the drawings, FIGS. 1-8 illustrate a spot occlusion apparatus 10 with opposing end effectors 24 in a spaced-apart opened state. The occlusion apparatus 10 of the invention includes an elongated tubular member 12, a pair of pincer occluding fingers 14 leading to end effectors 24, and an actuating device 16. The tubular member 12 extends along a central longitudinal axis “A” to define a lumen 18. The tubular member 12 has a distal end 20 and a proximal end 22 which is disposed opposite the distal end 20.

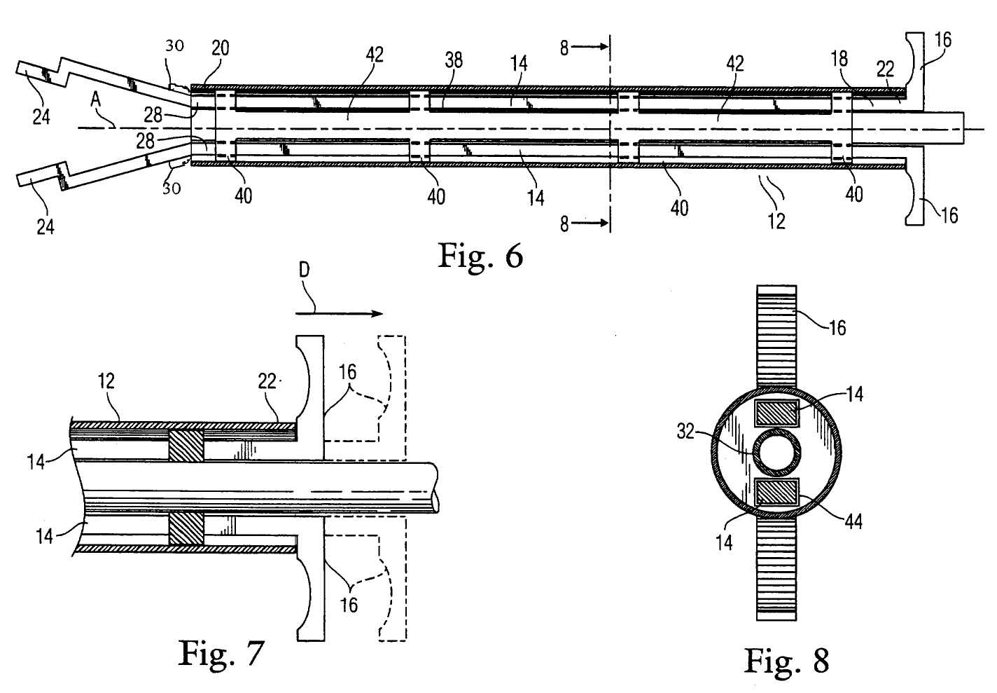

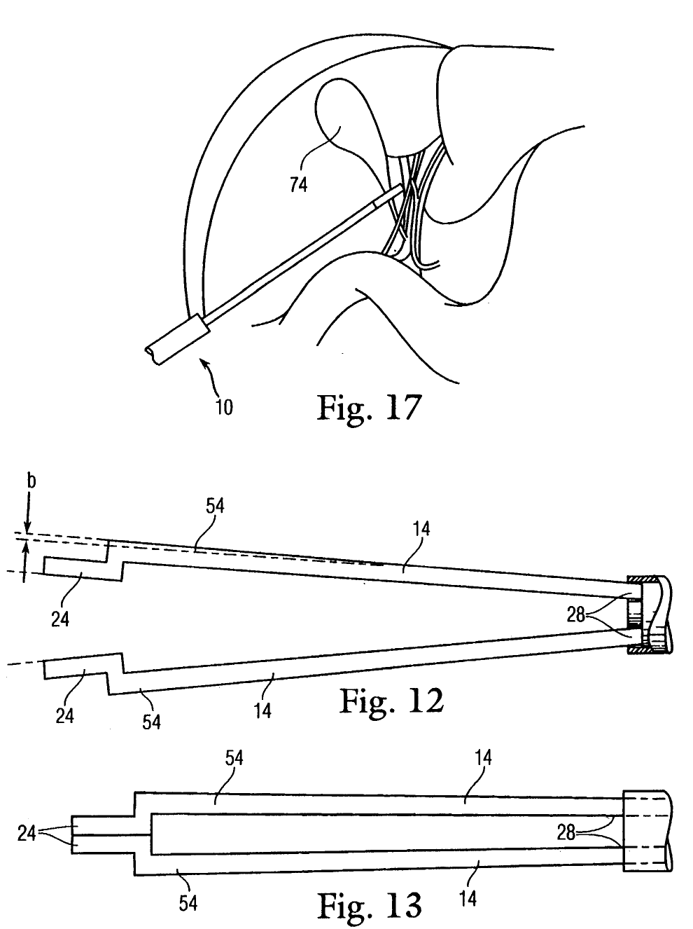

As best shown in FIG. 6, the pair of occluding fingers 14 extend into and through the lumen 18 and project generally longitudinally from the distal end 20 of the tubular member 12 in a facially-opposing relationship. The occluding fingers 14 are operative at the distal end of the tubular member 12 and wield the end effectors 24 disposed opposite one another. Thus, the end effectors 24 are moveable to and between an opened state and a closed state. In the opened state, the vessels to be occluded are received between the pair of end effectors 24. In the closed state, the pair of fingers 14/end effectors 24 flare outward and converge together either to straddle healthy tissue, or to create an enclosed working space for manipulating, coagulating and occluding targeted tissue. The tubular member 12 also encloses a central operating channel 34 (which may enclose/define one or more central ports directed into the workspace . . . through the same puncture) for admission and viewing by a viewing system, ingress of a hydration device, cauterization device, cryo (cooling) device, or any of a range of ancillary surgical implements all through a single surgical site. This configuration provides a significant advantage in that it creates a workspace within the fingers 14 which allows the surgeon to view and manipulate. Four or more different operating channels may be provided through central operating channel 34 to administer suction, irrigation, coagulating, and/or a viewing system. Traditional 3-puncture techniques can be completed with two punctures, four puncture techniques can be completed with three, etc.

Referring again to FIGS. 1-8, each occluding end effector 24 is moveable relative to one another (in cooperation with the pair of occluding fingers 14) to and between an opened state as shown in FIGS. 1, 4 and 6, and a closed state as shown in FIGS. 2 and 5. In the opened state, the occluding end effectors 24 are spaced apart from one another. In the closed state, the occluding end effectors 24 contact one another. However, as discussed below, the occluding end effectors 24 can be positioned adjacent to one another in the closed state.

As shown in FIGS. 1, 2, 6 and 7, the actuating device 16 is disposed at the proximal end 22 of the tubular member 12. The actuating device 16 is operative in conjunction with the occluding fingers 14 to move the occluding end effectors 24 to and between the opened and closed states. Although not by way of limitation, the actuating device 16 are individual finger tabs that extend transversely to the central longitudinal axis “A” as best shown in FIG. 6. Preferably, respective ones of the finger tabs are integrally formed with respective ones of the occluding fingers 14. However, one skilled in the art would appreciate that other conventional types of actuating devices could be used to move the occluding end effectors to and between the opened and closed states.

As best shown in FIGS. 3, 4 and 6, each occluding finger 14 includes a bent section 28. Thus, the occluding fingers 14 bend outwardly at an acute angle “a” as measured relative to the central longitudinal axis “A.” Further, the occluding fingers 14 bend outwardly from the distal end 20 of the tubular member 12 to form a bent condition in respective ones of the occluding fingers 14. The outwardly-bent occluding fingers 14 are resiliently biased in the bent condition to retain the respective occluding end effectors 24 in the opened state. The outwardly-bent occluding fingers 14 may be fabricated from metal having shape-memory characteristics such as Nitinol®. Pulling the actuating device 16 in a direction “D” as shown in FIG. 7 moves the occluding end effectors 24 to the closed state (FIGS. 2 and 5) from the opened state (FIGS. 1, 4 and 6). Pulling the actuating device 16 pulls the pair of occluding fingers 14 into the lumen 18 at the bent section 28 thereby pulling the occluding end effectors 24 toward the distal end 20 of the tubular member 12. Also, pushing the actuating device 16 opposite to the direction “D” shown in FIG. 7 moves the occluding end effectors 24 to the opened state from the closed state. Pushing the actuating device 16 also pushes the pair of occluding fingers 14 outwardly from the lumen 18 thereby pushing the occluding end effectors 24 away from the distal end 20 of the tubular member 12. Thus, the device has three operative states: 1) fingers 14 are outstretched outwardly from the lumen 18 and in the opened state; 2) fingers 14 are outstretched and in the closed state to create space and define the surgical workspace; 3) fingers 14 are retracted into the lumen 18.

In FIGS. 1 and 3-6, actuation of the pair of occluding fingers 14 into the closed state is assisted by ramped protuberances 30. Each ramped protuberance 30 is disposed adjacent the bent section 28. Also, each ramped protuberance 30 is disposed exteriorly of the lumen 18 when the occluding end effectors 24 are in the open state. Also, the respective protuberances 30 contact the distal end 20 of the tubular member 12 within the lumen 18 when the occluding end effectors 24 are in the closed state. The ramped protruberance 30 bears against the distal end 20 of the tubular member 12 to close the occluding end effectors 24 according to a profile. Specifically, the ramped protruberance 30 is steeper initially to effect a quicker closure per degree offset of the actuating device 16. Conversely, the ramped protruberance 30 levels out to give a greater degree of closure control per degree offset of the actuating device 16 when the end effectors 24 are nearly closed.

The occlusion apparatus 10 of the invention also includes an inner tubular member 32 that is disposed within the lumen 18 of the tubular member 12. The inner tubular member 32 defines a central operating channel 34 interiorly of the inner tubular member 32 and an annular channel 36 that is formed between the tubular member 12 and the inner tubular member 32. In other words, with the inner tubular member 32 disposed within the tubular member 12, the lumin 18 is divided into the central operating channel 34 and the annular channel 36 disposed about the operating channel 34. As shown in FIGS. 1-6, the pair of occluding fingers 14 are disposed within the annular channel 36.

Further, the occlusion apparatus 12 of the invention includes a frame structure 38 as best shown in FIGS. 3 and 6. The frame structure 38 includes a plurality of spacers 40 interconnected by a plurality of spacer has 42. The frame structure 38 retains the inner tubular member 32 centrally about the central longitudinal axis “A.” Also, as best shown in FIG. 3, each spacer 40 has a pair of guide holes 44. Each guide hole 44 is sized and adapted to slideably receive a respective one of the occluding fingers 14. The spacers 40 and guide holes 44 provide support and proper alignment of the occluding fingers 14.

The pair of occluding fingers 14 may be slidably removed from the guide holes 44 of spacer 40 for replacement with fingers 14 having different configurations of the occluding end effectors 24. FIGS. 9-11 represent a variety of alternate configurations of the occluding end effectors 24. In FIG. 9, the occluding end effector 24 has a generally flattened, rectangular shape. In FIG. 10, the occluding end effector 24 is arcuate. Various other configurations of the occluding end effectors 24 may be used as desired. In all of the foregoing configurations, the pair of occluding fingers 14 is insulated up to the operative portion of the occluding end effector 24. This is necessary because the fingers 14 carry electrical current from an energy source 50 (to be described) and should be shielded from inadvertent touching of surrounding tissue.

As seen in FIG. 11, the ramped protuberance 30 may be replaced by tapering the entirety of each occluding finger 14. The tapered section 54 extends approximately between the occluding end effector 24 and the bent section 28. The tapered section 54 narrows at an angle “b” from the occluding end effector 24 towards the bent section 28. Like the protuberance 30, the tapered section 54 assists in moving the occluding end effectors 24 from the opened state to the closed state.

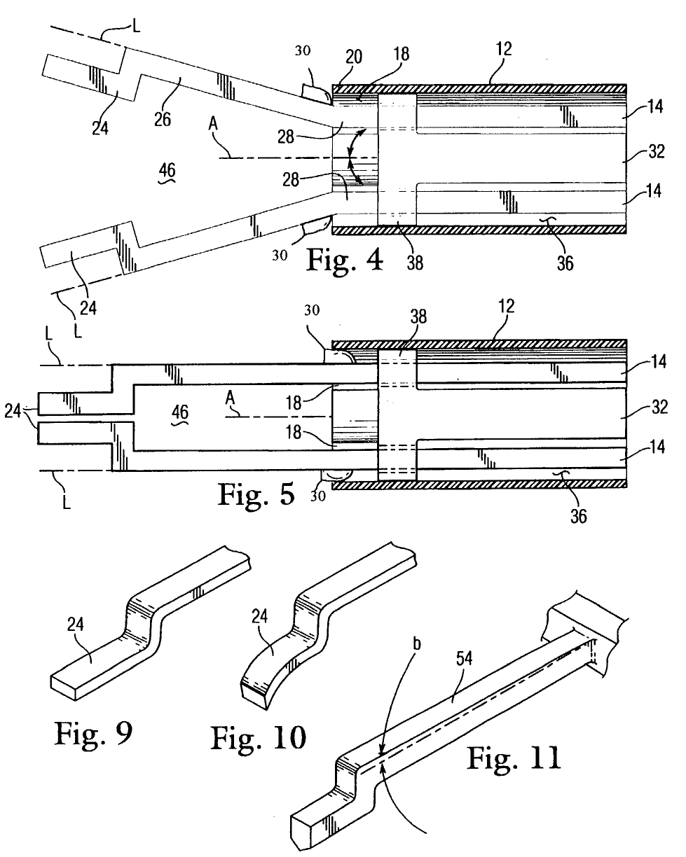

Referring back to FIGS. 4 and 5, the pair of occluding end effectors 24 extend parallel to respective longitudinal axes “L.” When the occluding end effectors 24 are in the closed state, the respective ones of the longitudinal axes “L” extend parallel to the central longitudinal axis “A.”

In FIGS. 4 and 5, an opening 46 is formed exteriorly of the distal end 20 of the tubular member 12 and between the pair of occluding fingers 14 for receiving anatomical tissue. When the occluding end effectors 24 are in the opened state (FIG. 4), the respective ones of the longitudinal axes “L” are disposed at the acute angle “a” relative to the central longitudinal axis “A.”

In FIG. 1, the occlusion apparatus 10 of the invention also includes a viewing system 48 and an energy source 50. The energy source is connected by wires to the respective occluding fingers 14 for conduction to the unshielded end effectors 24 (and on to the tissue). As is (known in the art, the viewing system 48 includes a viewing member (not shown) which is sized and adapted to be slidably received by operating channel 34 of the inner tubular member 32. Alternatively, the inner tubular member 32 itself could be the viewing member. For the occlusion apparatus shown in FIG. 1, respective ones of the occluding end effectors 24 are a pair of heating elements configured with a triangular cross-section. The energy source 50 is connected to terminals 52 to supply either electric energy, ultrasonic energy or laser energy to the pair of heating elements, particularly at the apex of each occluding end effector 24.

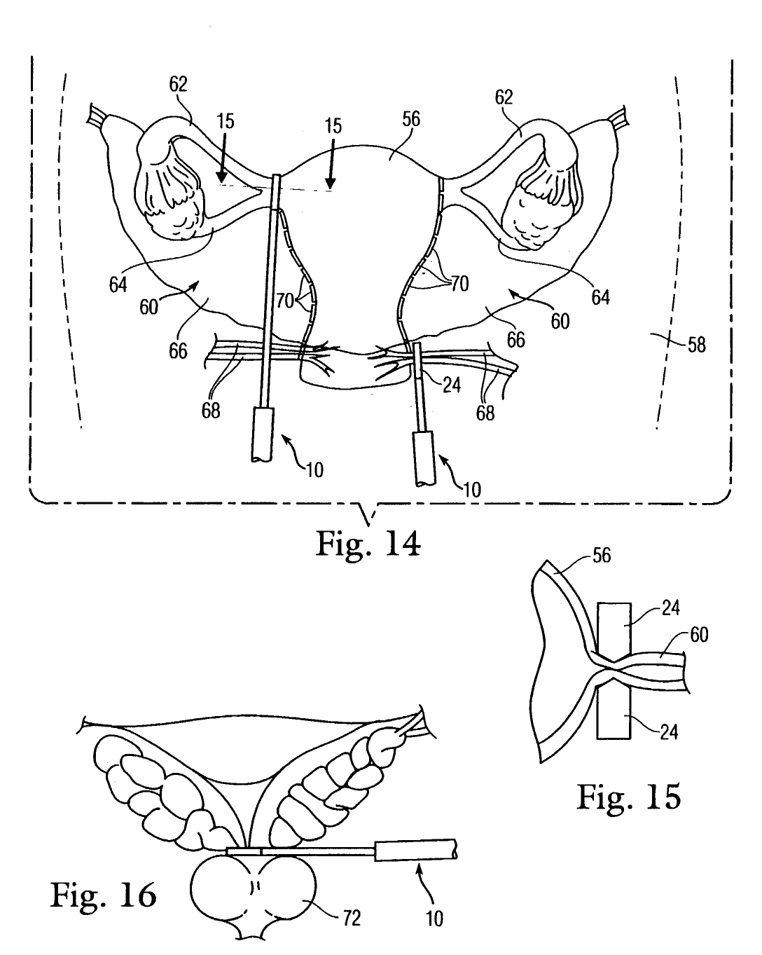

In use, the occlusion apparatus 10 of the invention is particularly useful for spot-coagulation and occlusion of an anatomical tissue structure such as a uterus 56. As shown in FIG. 14, the utems 56 is disposed in a living body 58. It should be noted that FIG. 14 is a perspective drawing for purposes of illustration of the occlusion apparatus 10. In practice, the device 10 would more likely be disposed frontally of the anatomical structure (the surgical procedure is typically not done from below due to lack of access). The anatomical tissue structure has a plurality of vessels that extend therefrom. As is known in the art, the uterus 56 is connected to and between a pair of ligamentous tissue structures 60 in the living female body 58. Generally, the ligamentous tissue structure 60 includes a pair of fallopian tubes 62, a pair of round ligaments 64, a pair of broad ligaments 66 and uterine vessels 68. A skilled artisan would appreciate that the ligamentous tissue structure 60 also includes a pair of ovarian ligaments (not shown,) a pair of uterosacrial ligaments and other tissues structures. The uterine vessels 68 are occluded adjacent the uterus 56 to cause ischemic necrosis of the uterus 56. As shown in FIG. 14, the occluding apparatus 10 of the invention is used for cauterizing the ligamentous tissue structure 60 adjacent the utems 56.

Specifically, cauterized effectors 70 are formed adjacent the uterus 56 on the fallopian tubes 62, the round ligaments 64, the broad ligaments 66 as well as the uterine vessels 68. However, a skilled artisan would appreciate that the uterine vessels 68 should be cauterized first and cauterization of the left and right ureters must be avoided.

FIG. 15 illustrates the pair of occluding end effectors 24 contacting and compressing the ligamentous tissue structure 60 and heating the same to achieve cauterization. However, occluding the ligamentous tissue structure 60 occurs without severing either one the ligamentous tissue structures 60. Thus, the uterus is still connected to the ligamentous tissue structure 60 even after performing the occlusion procedure. As a result of the occlusion procedure, the anatomical tissue structure, which in this case is the uterus 56, is rendered non-functional in situ.

As stated above, the occlusion apparatus 10 of the invention can be used with a variety of anatomical tissue structures. In FIG. 16, the occlusion apparatus 10 of the invention is used for occluding vessels connected to a prostate 72. In FIG. 17, the occlusion apparatus 10 of the invention is used for occluding vessels connected to a gall bladder 74.

For the occlusion apparatus 10 of the invention discussed above, it is appreciated that the occluding mechanism includes the energy source 50 and the pair of occluding end effectors 24 which operate as a pair of occluding elements. The energy source 50 is operative in conjunction with the pair of occluding elements to generate heat sufficient to cauterize (but not severe) the vessels, thereby occluding the anatomical tissue structure connected thereto. For the occlusion apparatus 10 of the invention, the energy source 50 can be electrical energy, ultrasound energy, laser energy or the like. A skilled artisan would appreciate that other occluding mechanisms can be used.

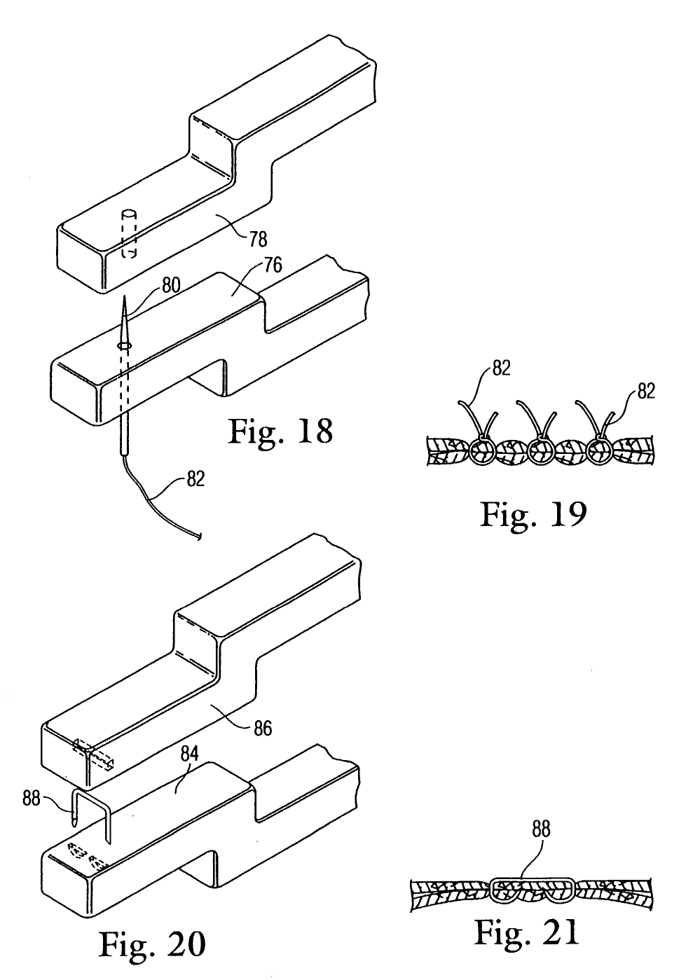

In FIG. 18, the occluding elements include a needle passer 76 and a needle catcher 78. Also, the occluding mechanism includes a needle 80 and a suture 82 connected to the needle 80. The needle passer 76 holds the needle 80 and suture 82 when the pair of occluding elements are in the open state. The needle catcher 78 catches the needs 80 when the pair of occluding elements are in the closed state. In FIG. 19, after the needle 80 and suture 82 are passed through vessels extending from anatomical tissue to be necrotized, the sutures 82 are tied. Thus, occlusion is achieved by suturing at least the vessels without severing the same. In FIG. 20, the occluding mechanism includes a pair of occluding elements in the form of an anvil 84 a staple holder 86 and a staple 88. In conjunction with the anvil 84 and the staple holder 86, the staple 88 moves between a pre-stapled condition (FIG. 20) and a stapled condition (FIG. 21). The staple 88 is carried by the staple holder 86 in the pre-stapled condition when the pair of occluding elements are in the opened state. The occluding mechanism is operative because the staple 88 moves from the pre-stapled condition to the stapled condition when the pair of occluding elements move to the closed state. The staple 88 in the stapled condition occludes the vessels thereby occluding the anatomical tissue structure. Thus, occlusion is also achieved by stapling without severing the anatomical tissue structure.

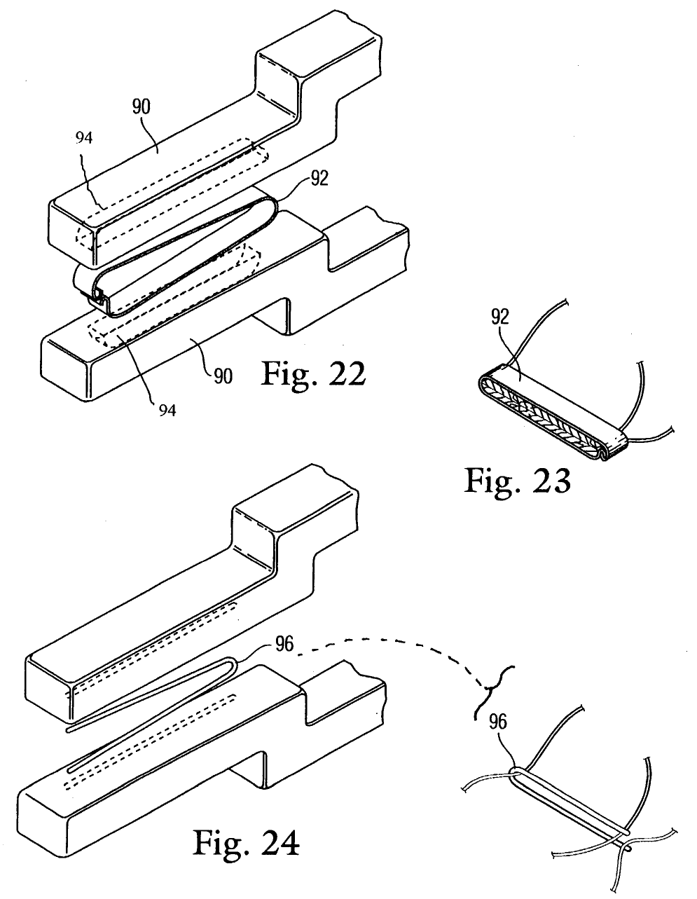

In FIGS. 22 and 23, the occluding mechanism includes a pair of occluding elements in a form of a pair of clamping members 90 and a clamp 92. Although not by way of limitation, at least one of the pair of clamping members 90 include a recess 94 that is sized and adapted for receiving and releasably holding the clamp 92. The clamp 92 moves from an opened condition (FIG. 22) to a closed condition (FIG. 23). The pair of occluding elements are configured to releaseably retain the clamp 92 in the opened condition so that the clamp 92 receives the vessels to be occluded. The pair of occluding elements are configured also to cause the clamp 92 to move to the closed condition to occlude the vessels, thereby occluding the anatomical tissue structure. Thus, occlusion is achieved by clamping without severing the anatomical tissue structure.

In FIG. 24, the occluding mechanism includes an occluding elements in a form of a pair of clamping members 90 which are similar to those discussed above. The occluding mechanism also includes a generally U-shaped clip 96 that moves from an opened condition to a closed condition. The pair of occluding elements are configured to releaseably retain the clip 96 in the opened condition so that the clip 96 receives the vessels to be occluded. The pair of occluding elements 90 are likewise configured also to cause the clip 96 to move to the closed condition (shown at right) to occlude the vessels, thereby occluding the anatomical tissue structure. Thus, occlusion is achieved by clamping without severing the anatomical tissue structure.

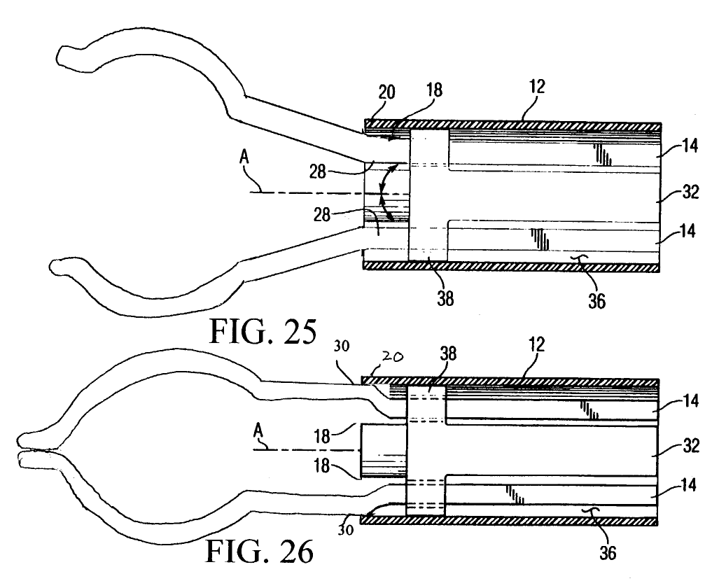

Yet another embodiment of the invention eliminates the need for ramped protuberances 30. This is accomplished by forming the occluding end effectors 24 in a pincer-like configuration and relying solely on the outward slant of the bent section 28 in the pair of occluding fingers 14 to fulfill the same function. FIGS. 25 and 26 illustrate this principle. As the outward slant of the bent section 28 in the pair of occluding fingers 14 contacts the distal end 20 of the tubular member 12 within the lumen 18 directly (moving from FIG. 25 to FIG. 26), the occluding fingers 14 them selves urge the occluding end effectors 24 into the closed state without any protruberances 30.

Note that none of the occluding mechanisms described result in severing the vessels. The anatomical tissue structure in each instance is spot-occluded without severing the vessels that are connected to them.

Another embodiment of the invention is a method for spot-coagulation and occlusion of an anatomical tissue structure in a living body. The anatomical tissue structure is connected to a plurality of vessels that extend from the anatomical tissue structure and convey fluids into and is out of the anatomical tissue structure. The method includes locating the anatomical tissue structure and the vessels extending therefrom to be spot-coagulated in the living body. A next step is spot-coagulating and/or otherwise occluding the vessels to prevent fluid flow into and out of the anatomical tissue structure to cause ischemic necrosis of the anatomical tissue structure.

In some instances, a tissue mass is connected to the anatomical tissue structure and supports the vessels extending from the anatomical tissue structure. As discussed above, by way of example only, the tissue mass can be the ligamentous tissue structure that supports the uterus and vessels extending therefrom. The vessels can be either embedded in the tissue mass or disposed on a surface of the tissue mass. The tissue mass itself, along with the vessels, can be occluded by cauterizing, stapling, clamping, clipping or suturing. The anatomical tissue structure is typically defined by an outer surface. Preferably, the step of occluding the vessels occurs adjacent the outer surface of the anatomical tissue structure, or the step of occluding the vessels occurs after isolating the vessels from the anatomical tissue structure. In order to isolate, the tips of occluding end effectors 24 should be sharp to penetrate the tissue structure. Preferably, the vessels include at least one blood-conveying artery and at least one blood-conveying vein. Such blood-conveying artery and blood-conveying vein are illustrated by way of example, only, in FIG. 14 as uterine vessels 68. The pair of occluding fingers 14 give the present device a pincer ability to reach around healthy tissue structures to the uterine arteries or other target structure in order to perform spot-coagulation and other surgical procedures. The device excels in close-quarter procedures such as occlusion (the alternative surgical procedure to a hysterectomy) because of the pincer-like fingers that straddle and reach around healthy tissue. Moreover, when the device is not straddling healthy tissue, the articulating fingers 14 create an enclosed working space for manipulating, coagulating and occluding targeted tissue. The one or more ports pass through the center of the device and enter the working space for admission of a viewing system, hydration device, cauterization device, cryo (cooling) device, or any of a range of ancillary surgical implements all to the single surgical site. The integral viewing system port eliminates the need for a separate puncture for the endoscope. Thus, the apparatus and method reduces surgery

Practicing the occlusion apparatus and method for occlusion of an anatomical tissue structure of the invention avoids removal of the anatomical tissue structure from the living body. Also, minimal invasive surgery techniques can be used to practice the invention. Benefits of practicing the invention include less hospital stay and less recovery time for the patient.

Although the embodiments of the invention have been specifically described herein, it would be apparent to those skilled in the art to which the invention pertains that other variations and modifications of the embodiments herein maybe made without departing from the spirit and scope of the invention.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come from within the meaning and range of equivalency of the claims are therefore intended to be embraced therein.

I claim:

1. A spot coagulating and occluding apparatus, comprising:
an elongated tubular member extending along a central longitudinal axis to define a lumen and having a distal end and a proximal end disposed opposite the distal end;
a pair of occluding fingers projecting generally longitudinally from the distal end of the tubular member in a facially-opposing relationship, each one of the pair of occluding fingers terminating in an occluding end effector with the occluding end effectors being movable relative to one another to and between an opened state wherein the occluding end effectors are spaced apart from one another and a closed state wherein the occluding end effectors are one of adjacent to and in contact with one another;
a central channel running through said tubular member between the pair of occluding fingers and opening outward between said end effectors;
whereby a proximal end of the tubular member is operative in conjunction with the occluding fingers to move the occluding end effectors to and between the opened and closed states.

2. An occlusion apparatus according to claim 1, wherein the actuating device moves the occluding end effectors to the closed state from the opened state by pulling the pair of occluding fingers into the lumen thereby pulling the occluding end effectors toward the distal end of the tubular member and moves the occluding end effectors to the opened state from the closed state by pushing the pair of occluding fingers outwardly from the lumen thereby pushing the occluding end effectors away from the distal end of the tubular member.

3. The spot coagulating and occluding apparatus according to claim 2, wherein each one of the pair of occluding fingers includes a protuberance adjacent the bent section and exteriorly of the lumen when the occluding end effectors are in the opened state, the protuberance contacting the tubular member within the lumen when the occluding end effectors are in the closed state.

4. The spot coagulating and occluding apparatus according to claim 2, wherein each one of the pair of occluding fingers includes a tapered section extending between the occluding end effector and the bent section, the tapered section narrowing from the occluding end effector towards the bent section.

5. The spot coagulating and occluding apparatus according to claim 1, further comprising an inner tubular member disposed within the lumen of the tubular member to define a central channel interiorly of the inner tubular member and an annular channel formed between the tubular member and the inner tubular member, the pair of occluding fingers being disposed within the annular channel.

6. The spot coagulating and occluding apparatus according to claim 5, wherein the pair of occluding end effectors define respective longitudinal axes and, when the occluding end effectors are in the closed state, the respective ones of the longitudinal axes extend parallel to the central longitudinal axis and, when the occluding end effectors are in the opened state, the respective ones of the longitudinal axes are disposed at an acute angle relative to the central longitudinal axis.

7. The spot coagulating and occluding apparatus according to claim 5, further comprising a viewing system including a viewing member sized and adapted to be slidably received by the inner tubular member.

8. The spot coagulating and occluding apparatus according to claim 1, wherein respective ones of the occluding end effectors are interchangeable ones of a pair of heating elements, an anvil and staple holder, a pair of clamp holders, a pair of clip holders and a needle passer and needle catcher.

9. The spot coagulating and occluding apparatus according to claim 1, wherein respective ones of the occluding end effectors are interchangeable ones of a pair of heating elements, an anvil and staple holder, a pair of clamp holders, a pair of clip holders biopsy forceps, scissors, and a needle passer and needle catcher.

10. The spot coagulating and occluding apparatus according to claim 1, wherein said central channel is adapted for passing one or more surgical instruments into a space between said end effectors for access to tissue held thereby.

11. The spot coagulating and occluding apparatus according to claim 8, further comprising an energy source operative with the pair of heating elements.

12. The spot coagulating and occluding apparatus according to claim 9, wherein the energy source is one of electric energy and vibrational energy.

13. The spot coagulating and occluding apparatus according to claim 1, wherein the pair of occluding finger members extend into and through the lumen.

14. The spot coagulating and occluding apparatus according to claim 13, wherein each one of the pair of occluding fingers includes a bent section wherein the occluding fingers bend outwardly at an acute angle relative to the central longitudinal axis from the distal end of the tubular member to form a bent condition, the outwardly-bent occluding fingers being resiliently biased in the bent condition to retain the respective occluding end effectors in the opened state.

15. A surgical instrument, comprising:
an elongated tubular member extending along a central longitudinal axis to define a lumen and having a distal end and a proximal end disposed opposite the distal end;
a pair of bent fingers projecting generally longitudinally from the distal end of the tubular member, each finger bending around to an end effector, and the fingers being movable relative to one another to and between an opened state wherein the end effectors are spaced apart from one another and a closed state wherein the end effectors are together and the bent fingers define a surgical workspace therebetween;
a central channel running through said tubular member between the pair of occluding fingers and opening outward between said end effectors;
an actuating handle disposed at the proximal end of the tubular member and operative to manually move the end effectors to and between the opened and closed states.

16. The surgical instrument according to claim 15, wherein said elongated tubular member further comprises a central operating channel opening between said fingers.

17. The surgical instrument according to claim 15, wherein said central operating channel defines a central port directed into the workspace.

18. The surgical instrument according to claim 16, wherein said central channel channel defines a plurality of ports directed into the workspace.

19. The surgical instrument according to claim 18, wherein said plurality of ports directed into the workspace allow admission of any one from among a group consisting of a viewing system, hydration device, cauterization device, suction device, and cooling device.

20. The surgical instrument according to claim 15, wherein said pair of bent fingers are articulable in any one from among three operative states, including an open state in which said fingers are outstretched outwardly from the lumen, a closed state in which said fingers are outstretched and closed to define the surgical workspace, and a retracted state in which the fingers are retracted into the lumen.

21. The surgical instrument according to claim 20, wherein said pair of bent fingers are formed of Nitinol®.

22. The surgical instrument according to claim 20, wherein each one of the pair of fingers includes a bent section wherein the fingers bend outwardly at an acute angle relative to the central longitudinal axis from the distal end of the tubular member to form a bent condition, the outwardly-bent fingers being resiliently biased in the bent condition to retain the respective occluding end effectors in the opened state.

23. A spot coagulating and occluding apparatus, comprising:
a tubular member extending along a central longitudinal axis to define a lumen and having a distal end and a proximal end disposed opposite the distal end;
a pair of occluding fingers projecting from the distal end of the tubular member, flaring outward, and converging together in an opposing relationship, each one of the pair of occluding fingers terminating in an occluding end effector with the occluding end effectors being movable relative to one another to and between an opened state wherein the occluding end effectors are spaced apart from one another and a closed state wherein the fingers flare outward from said tubular member to straddle healthy tissue and then converge together into contact with one another in order for said end effectors to operatively occlude targeted tissue;
whereby a proximal end of the tubular member is operative in conjunction with the occluding fingers to move the occluding end effectors to and between the opened and closed states.

24. The occlusion apparatus according to claim 23, further comprising a central channel running through said tubular member between the pair of occluding fingers and opening outward between said end effectors.

25. The spot coagulating and occluding apparatus according to claim 23, further comprising an actuating device that moves the occluding end effectors to the closed state from the opened state by pulling the pair of occluding fingers into the lumen thereby pulling the occluding end effectors toward the distal end of the tubular member and moves the occluding end effectors to the opened state from the closed state by pushing the pair of occluding fingers outwardly from the lumen thereby pushing the occluding end effectors away from the distal end of the tubular member.
